(19) United States
(12) Patent Application Publication
(10) Pub. No.: US 2026/0209149 A1
(43) Pub. Date: Jul. 23, 2026

(54) SEPARATION PROCESS OF ALCOHOL MIXTURES WITH HIGH WATER CONTENT BY LIQUID-LIQUID EXTRACTION

(71) Applicant: ENI S.P.A., Roma (IT)

(72) Inventors: Laura Annamaria PELLEGRINI, Milano (IT); Elvira SPATOLISANO, Milano (IT); Samuele GORI, Roma (IT); Giacomo FILIPPINI, Roma (IT)

(73) Assignee: ENI S.P.A., Roma (IT)

(21) Appl. No.: 19/135,823

(22) PCT Filed: Dec. 4, 2023
(86) PCT No.: PCT/IB2023/062170

§ 371 Date: Jun. 5, 2025

(30) Foreign Application Priority Data
Dec. 6, 2022 (IT) 102022000025080

(51) Int. Cl. C07C 29/86; B01D 11/04; C07C 31/08; C07C 31/10

(52) U.S. Cl. CPC C07C 29/86; B01D 11/0492; C07C 31/08; C07C 31/10

(57) ABSTRACT

The present invention relates to a process for separating alcohol mixtures with high water content using naphtha as an extraction solvent.

In particular, the present invention is directed to a process for producing a biofuel comprising or consisting of a step of liquid-liquid extraction of a mixture of C2-C3/C2-C4 alcohols from a mixture of C2-C3/C2-C4 alcohols and water with an extraction solvent, where the extraction solvent is a naphtha.

### TECHNICAL FIELD OF THE INVENTION

The present invention relates to a process for separating alcohol mixtures with high water content.

### Background Art

While water-alcohol separation is a widely known and studied problem in the literature, the separation of alcohol mixtures from water (i.e., when multiple alcohol species are simultaneously present in an aqueous solution) is a more rarely addressed problem due to the complexity thereof. The difficulty in the separation lies in the identification of a solvent or solvent mixture capable of simultaneously extracting all the alcohols, separating them from the significant water content in which they are dissolved, while minimizing the energy demands of the process.

Ethylene glycol is one of the most widely used solvents in the context of extractive water-alcohol distillation. The high boiling point and high water affinity make ethylene glycol an excellent dehydrating agent. The solution usually adopted to promote the dehydration of a stream of alcohols can be summarized as follows: the added solvent (i.e., ethylene glycol) is fed, together with the water-alcohol mixture to be separated, to an extractive distillation column. Such a column carries out the fractionation of the alcohol stream, obtained as a top product, from the water-ethylene glycol stream, recovered from the bottom of the aforesaid column. The latter stream must be further fractionated in a downstream distillation column, so that the solvent can be regenerated and then recycled to the extractive distillation column. Extractive distillation with ethylene glycol as a solvent usually requires a non-negligible thermal load to be provided to the reboiler of the solvent regeneration column.

In fact, despite the chemical-physical features of ethylene glycol making it particularly suitable for applications as a dehydrating agent, the high boiling character thereof is responsible for the high temperatures reached during regeneration, which evidence weighs down the energy requirements of the process itself (i.e., the thermal loads of the column bottom reboilers and the related operating temperatures).

As an alternative to extractive distillation, liquid-liquid extraction using toluene, iso-octane or other hydrocarbon species as solvents is reported in the literature. However, the suggested solutions concern negligible amounts of mixed water and ethanol. Therefore, they are not flexible as for the distribution of alcohols in the feed and are not suitable for the separation of alcohol mixtures particularly diluted in water.

Various known processes provide mixtures of alcohols, in particular C2-C3 alcohols, in water as products or by-products. An example is the glycerol hydrogenation process to give propanols described in co-pending patent application No. 102021000016859 filed on 28 Jun. 2021.

In particular, the use of propanols in biofuels is recommended. Propanols have the same positive features as ethanol with reference to the ignition qualities and calorific value thereof, but have higher energy density (+12%), lower volatility, and lower latent vaporization heat (−8%). In principle, a good source of propanols can be glycerin.

Propanols can also be produced using noble metal-based catalysts, modified by dopants and promoters, in particular metal oxides (groups 4-7) (Shinmi, Y., Koso, S., Kubota, T., Nakagawa, Y., Tomishige, K., Modification of Rh/SiO₂ catalyst for the Hydrogenolysis of Glycerol in Water, Appl. Catal. B Environ. 2010, 94 (3-4), 318-326).

These processes produce mixed propanols with a high amount of water which can reach up to 50% by weight and, as such, cannot be advantageously used in a biofuel.

Therefore, there is a need to provide a simple and cost-effective process for isolating a substantially water-free mixture of propanols.

### SUMMARY OF THE INVENTION

The present invention is based on the discovery that treating a mixture of C2-C4 alcohols and water with a naphtha yields a substantially water-free naphtha enriched in C2-C3 alcohols which can be used as such as a fuel.

With the term “naphtha” it is herein identified the “petroleum naphtha”, i.e. a fraction of crude oil which mainly comprises (% by volume) linear and cyclic aliphatic hydrocarbons having a boiling point between 125° C. to 240° C. (including the end points).

Therefore, the present invention relates to:

1) a process for producing a biofuel comprising or consisting of a step of liquid-liquid extraction of a mixture of C2-C3 alcohols from a mixture of C2-C3 alcohols and water with an extraction solvent, where the extraction solvent is a naphtha.

The invention is also directed to:

2) a process according to 1), where the mixture of C2-C3 alcohols consists of ethanol, n-propanol, and isopropanol;

3) a process according to 1) or 2), where the water content in the mixture of C2-C3 alcohols and water is up to 50% by weight;

4) a process according to any one of 1) to 3), where naphthas are selected from reforming naphtha (RC3 and RC2), alkylation naphtha, isomerization naphtha, FCC naphtha, and Light Cracked Naphtha (LCN);

5) a process according to any one of 1) to 3), where naphthas have a weight ratio of aromatics-olefins/paraffins between 0.5 and 4, preferably between 0.6 and 3.8;

6) a process according to any one of 1) to 5), where the recovery of C2-C3 alcohols is higher than 95% by weight, preferably higher than 99% by weight;

7) a process according to any one of 1) to 6), where the weight ratio of extraction solvent to fed water-alcohol mixture is between 2:1 and 10:1, preferably between 4:1 and 8:1;

8) a process according to any one of 1) to 7), where the process is conducted at ambient pressure and temperature;

9) a process according to any one of 1) to 8), where the process is conducted in a liquid-liquid separator, where the extraction solvent is fed into the lower section and the mixture of C2-C3 alcohols and water is fed into the upper section, and where the extracted mixture of naphtha and C2-C3 alcohols is removed in the upper section and the separated water is removed in the lower section.

### BRIEF DESCRIPTION OF THE DRAWINGS

FIG. 1 shows a simplified diagram illustrating the process of the invention in a preferred embodiment.

### DETAILED DESCRIPTION OF THE INVENTION

The invention aims to perform a separation of a mixture of short-chain primary alcohols (mainly C2-C4, preferably C2-C3) containing a high water content (up to about 50% by weight) using an extracting agent selected from naphthas.

The present invention relates to a process for producing a biofuel comprising or consisting of a step of liquid-liquid extraction of a mixture of C2-C4 or C2-C3 alcohols from a mixture of C2-C3/C2-C4 alcohols and water (hereinafter also referred to as the “water-alcohol mixture”) with an extraction solvent, where the extraction solvent is a naphtha.

For the purposes of the present invention, the term “comprising” means a process where the characterizing step consists of the liquid-liquid extraction, but which can also include other steps such as a pre-treatment of the mixture of C2-C3/C2-C4 alcohols in water or the final addition of additives to the organic mixture forming the extract.

For the purposes of the present invention, the term “consisting of” means a process where no further post-processing of the extract is included, i.e., where the extract containing naphtha and C2-C3/C2-C4 alcohols is used as such as a biofuel.

Different classes of mineral naphtha can be used as the extraction solvent.

In Italy, the physical and chemical properties of petrol and naphtha are governed by regulations issued by CUNA (Commissione tecnica di UNificazione nell'Autoveicolo-Technical Commission for Unification in Motor Vehicles). The most important properties are anti-knock power, expressed by the octane number, and volatility. Car petrol boils between 35 and 210° C. as it contains hydrocarbons with a number of carbon atoms between 4 and 8. Topping petrol is referred to Primary Petrol. Other refining processes were then adopted, such as catalytic cracking, thermal and catalytic reforming, polymerization, alkylation, isomerization, from which products such as reformed naphtha, alkylated naphtha, isomerized naphtha, etc., are derived.

Synonyms of the term “naphtha” as used in this patent application can be “crude oil”, “fuel oil” or “diesel oil” (semi-dense naphthas), “kerosene” or “diesel oil” (light naphthas with cetane number between 40 and 75).

In the refinery industry, the various types of naphtha are commonly mixed in different percentages to obtain the desired product used in the automotive field (e.g., RON95 petrol).

The invention allows obtaining an extract containing a mixture of alcohols as an octane booster directly usable in the mixture of the finished product with better motor properties (e.g., octane number) than the starting naphtha. Given that the production capacities (kton/year) of the different naphthas are peculiar to each refinery, the process of the invention is applicable to the following naphthas: reforming naphtha (RC3 and RC2), alkylation naphtha, isomerization naphtha, FCC naphtha and Light Cracked Naphtha (LCN).

The following table 1 shows the composition of some naphthas which can be used as an extraction solvent for C2-C3/C2-C4 alcohols according to the invention.

TABLE 1
Composition and molecular weight (MW) of solvents considered
for liquid-liquid extraction depicted in Error! Reference source not found.

| | | | | | FCC- | FCC- | |
|---|---|---|---|---|---|---|---|
| | RC3 | RC2 | alkylated | isomerized | 2017 | 2022 | LCN |
| Paraffins | 1.28 | 1.75 | 0.54 | 15.69 | 9.59 | 11.37 | 0.69 |
| cycle | | | | | | | |
| Paraffins | 24.19 | 20.4 | 97.13 | 83.8 | 36.03 | 41.11 | 43.9 |
| Olefins | 0.08 | 0.1 | 0.07 | 0.09 | 4.63 | 3.69 | 1.94 |
| cycle | | | | | | | |
| Olefins | 0.76 | 0.5 | 0.65 | 0.26 | 25.41 | 18.7 | 53.45 |
| Aromatics | 73.69 | 77.23 | 1.29 | 0.18 | 24.35 | 25.14 | 0.02 |
| MW | 102.9871 | 104.5683 | 109.9995 | 78.8016 | 92.8040 | 93.5279 | 72.7136 |
| [kg/kmol] | | | | | | | |

The mixture of C2-C3 alcohols comprises ethanol, n-propanol, and isopropanol as main components.

An example of a typical composition of a water-alcohol mixture to be subjected to the process of the invention is shown below:

Mass fraction

| | |
|---|---|
| Water | 0.4747 |
| Ethanol | 0.0271 |
| Propanol | 0.3946 |
| Isopropanol | 0.1035 |
| F [kg/h] | 34798.9227 |

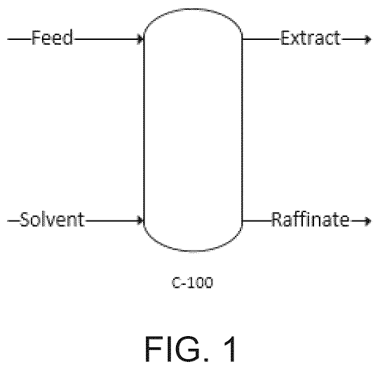

The solvent, selected from those shown in table 1, is fed from below into a liquid-liquid extraction column (C-100 in FIG. 1) operating at atmospheric pressure and ambient temperature, in which it is contacted with the water-C2-C3/C2-C4 alcohol feed to be separated, introduced into the upper section of the separator. Such an apparatus allows the separation of the aqueous phase (Raffinate in Error! Reference source not found. 1) from the organic phase (Extract in Error! Reference source not found.). The organic phase, which contains the solvent (naphtha) in which almost all of the alcohols present in the mixture are dissolved, is removed from the upper section of the separator, while the aqueous phase, which consists of water containing traces of alcohols and added solvent, is removed from the lower section. The organic phase forms the ready-to-market biofuel mixture.

The weight ratio of solvent to water-alcohol mixture is between 2:1 and 10:1, preferably between 4:1 and 8:1.

Tables 2 and 3 show the results of the liquid-liquid extraction according to the invention using two different solvent/water-alcohol mixture weight ratios (S/Mix ratio). Data in table 2 refer to a S/mix ratio=4, while those in table 3 refer to a S/mix ratio=8.

TABLE 2
Performance for the process diagram shown in 1 as the naphtha
composition considered varies. The S/Mix ratio entering
the extractor 4[kg/kg]. The residual water content
(H₂O) is calculated with respect to alcohols alone.

| | | Bio/fossil | ThOD | Alcohol |
|---|---|---|---|---|
| | H2O | in Extract | Raffinate | recovery |
| | [ppmwt] | [% wt] | [g/L] | [wt %] |
| RC3 | 44141.75 | 12.82 | 62.26 | 97.5955 |
| RC2 | 42557.56 | 12.83 | 59.32 | 97.6750 |
| alkylated | 54497.44 | 6.97 | 773.41 | 53.0194 |
| isomerized | 51296.4 | 12.22 | 184.46 | 92.9795 |
| FCC-2017 | 57360.05 | 12.68 | 106.56 | 96.4905 |
| FCC-2022 | 53480.03 | 12.60 | 117.86 | 95.8655 |
| LCN | 85055.77 | 12.87 | 96.73 | 97.8747 |

TABLE 3
Performance for the process diagram shown in 1 as the naphtha
composition considered varies. The ratio S/Mix entering
the extractor 8[kg/kg]. The residual water content
(H₂O) is calculated with respect to alcohols alone.

| | | Bio/fossil | ThOD | Alcohol |
|---|---|---|---|---|
| | H₂O | in Extract | Raffinate | recovery |
| | [ppmwt] | [ % wt] | [g/L] | [wt % ] |
| RC3 | 50639.51 | 6.52 | 18.53 | 99.3381 |
| RC2 | 48462.73 | 6.52 | 16.92 | 99.3705 |
| alkylated | 55363.75 | 6.27 | 114.55 | 95.4420 |
| isomerized | 60509.10 | 6.43 | 62.96 | 97.9483 |
| FCC-2017 | 64630.59 | 6.50 | 41.02 | 99.0127 |
| FCC-2022 | 60588.51 | 6.49 | 41.61 | 98.8444 |
| LCN | 96250.88 | 6.53 | 54.73 | 99.3852 |

In the aforesaid tables 2 and 3, the “bio/fossil” ratio is the percentage by weight of propanols+ethanol in the extract, with respect to the percentage by weight of naphtha in the extract.

From the data reported in the tables, it is noted that the best results are obtained with naphthas in which the weight ratio of aromatics-olefins/paraffins is between 0.5 and 4, preferably between 0.6 and 3.8. In such cases, the process of the invention allows obtaining a recovery of C2-C3 alcohols greater than 95% by weight. When naphthas in which the ratio of aromatics-olefins/paraffins is less than 0.5, in particular less than 0.1, are instead used, a recovery of C2-C3 alcohols greater than 95% by weight is only obtained using an S/Mix ratio greater than 7.

The process of the invention allows achieving several advantages.

The process of the invention, which consists of a unitary operation, has the advantage of promoting the separation between the aqueous phase and the organic phase with negligible energy costs, with the matter exchange occurring in the liquid phase only. Therefore, the subsequent and expensive stage of regenerating the solvent itself is avoided, introducing a considerable simplification of the process diagram which includes, in the present case, a single extraction column.

1. A process for producing a biofuel comprising or consisting of a step of liquid-liquid extraction of a mixture of C2-C3 or C2-C4 alcohols from a mixture of C2-C3/C2-C4 alcohols and water with an extraction solvent, wherein the extraction solvent is a naphtha.

2. The process according to claim 1, wherein the mixture of C2-C3 alcohols consists of ethanol, n-propanol and isopropanol.

3. The process according to claim 1, wherein the water content in the mixture of C2-C3 alcohols and water is up to 50% by weight.

4. The process according to claim 1, wherein naphthas are selected from reforming naphtha (RC3 and RC2), alkylation naphtha, isomerization naphtha, FCC naphtha, and Light Cracked Naphtha (LCN).

5. The process according to claim 1, wherein naphthas have a weight ratio of aromatics-olefins/paraffins between 0.5 and 4, preferably between 0.6 and 3.8.

6. The process according to claim 1, wherein the recovery of C2-C3 alcohols is higher than 95% by weight, preferably higher than 99% by weight.

7. The process according to claim 1, wherein the weight ratio of extraction solvent to fed mixture of water-alcohols is between 2:1 and 10:1, preferably between 4:1 and 8:1.

8. The process according to claim 1, wherein the process is conducted at ambient temperature and pressure.

9. The process according to claim 1, wherein the process is conducted in a liquid-liquid separator, where the extraction solvent is fed into the lower section and the mixture of C2-C3/C2-C4 alcohols and water is fed into the upper section, and wherein the extracted mixture of naphtha and C2-C3/C2-C4 alcohols is removed in the upper section and the separated water is removed in the lower section.
